(19) United States
(12) Patent Application Publication
(10) Pub. No.: US 2020/0069908 A1
(43) Pub. Date: Mar. 5, 2020

(54) PINEAL GLAND STIMULATION SYSTEM AND METHOD

(71) Applicant: Roderick Rutledge, Panama City Beach, FL (US)

(72) Inventor: Roderick Rutledge, Panama City Beach, FL (US)

(21) Appl. No.: 16/560,978

(22) Filed: Sep. 4, 2019

Related U.S. Application Data
(60) Provisional application No. 62/726,935, filed on Sep. 4, 2018.

(51) Int. Cl. A61M 21/02; A61H 21/00; A61H 23/02

(52) U.S. Cl. CPC A61M 21/02; A61M 2021/0022; A61H 23/02; A61H 21/00

(57) ABSTRACT

A pineal gland vibration unit which may be designed to fit in the roof of the mouth of a user. The system may be designed to vibrate at a frequency which is pleasing to the senses. The system may be wirelessly operated. The system may induce spiritual awareness while meditating with the system installed. The device may further allow a user to meditate and become more aware of oneself during self-healing processes.

### CROSS-REFERENCE TO RELATED APPLICATION(S)

The present application is related to and claims priority to U.S. Provisional Patent Application No. 62/726,935 filed Sep. 4, 2018, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of dentistry of existing art and more specifically relates to dentistry including extra-oral force transmitting means.

### RELATED ART

Modern society demands can be very stressful. People may enjoy meditating and being spiritual to deal with stress effectively and for heightened awareness of themselves and their surroundings. Self-awareness and self-healing can be crucial for some people's positive mental well-being. The pineal gland may be an internal organ capable of promoting better self-awareness and spirituality; however, activating the pineal gland may be difficult. A suitable solution is desired.

U.S. Pat. No. 9,352,157 to Hui Tang relates to an intra-oral balance device based on palatal stimulation. The described intra-oral balance device based on palatal stimulation includes a device for vestibular substitution with a mouthpiece fitting entirely within a person's mouth in a shape that conforms to the palate. The mouthpiece encases a circuit board containing an electronic system that can deliver electrical pulses to electrical stimulators touching the palate based on head movement. The electronic system includes a motion sensor, a control unit; a stimulation circuit; and a battery. The control unit is preferably a microcontroller with a digital Input/Output capability, a Serial Peripheral Interface/Inter-Integrated Circuit protocol, a timer, and an oscillator for sensor interfacing and timing control. The microcontroller enables processing data from the accelerometer to indicate head movement and controlling each embedded electrical stimulator to deliver electrical pulses with adjustable waveform parameters. The device may also include a wireless transceiver for remote control of the device from outside the person's mouth

### SUMMARY OF THE INVENTION

In view of the foregoing disadvantages inherent in the known dentistry art, the present disclosure provides a novel pineal gland stimulation system and method. The general purpose of the present disclosure, which will be described subsequently in greater detail, is to provide an effective means to stimulate the pineal gland to promote meditative and spiritual ambiance.

The present invention provides users with a vibrating device that may be capable of opening the pineal gland to improve meditation and spirituality. The device vibrates at various frequencies when inserted in the mouth and applied to the roof of the mouth, effectively triggering the pineal gland. It offers a way for users to become more self-aware during self-healing processes, meditation, and the like. The present invention allows users to be influenced by spirituality and self-awareness in various ways by triggering the pineal gland.

A pineal gland stimulation system is disclosed herein, the system comprising: an oral mouth-piece assembly including a body contoured to fit to a roof of a mouth of a user-wearer having an outer surface; and an interior (inner) volume; a vibration mechanism in communication with an external electronic device such that signals can be sent between the vibration mechanism and the external electronic device to control vibration frequency of the vibration mechanism; and a battery for powering the vibration mechanism. As designed, vibrations of the vibration mechanism, when activated, trigger the pineal gland of the user-wearer between the two hemispheres of the brain of the user-wearer.

A method of use for the pineal gland stimulation system is also disclosed herein, the method comprising the steps of: providing an oral mouth-piece assembly including a body contoured to fit to a roof of a mouth of a user-wearer having an outer surface; and an interior volume; a vibration mechanism in communication with an external electronic device such that signals can be sent between the vibration mechanism and the external electronic device to control vibration frequency of the vibration mechanism; and a battery for powering the vibration mechanism; next inserting the oral mouth-piece assembly into the mouth of the user-wearer behind teeth and on the roof of the mouth; followed by turning the vibration mechanism to an on-condition via the external electronic device; then activating the vibration mechanism to vibrate, that when activated triggers a pineal gland of the user-wearer between two hemispheres of a brain of the user-wearer; and next adjusting a frequency of vibration via the external electronic device; wherein the oral mouth-piece assembly fits entirely within the mouth of the user-wearer and behind teeth of the user-wearer during use.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a pineal gland stimulation system, constructed and operative according to the teachings of the present disclosure.

FIG. 1 is a perspective view of the pineal gland stimulation system during an ‘in-use’ condition, according to an embodiment of the disclosure.

FIG. 2 is a perspective view of the pineal gland stimulation system of FIG. 1, according to an embodiment of the present disclosure.

FIG. 3 is a perspective view of the pineal gland stimulation system of FIG. 1, according to an embodiment of the present disclosure.

FIG. 4 is a perspective view of the pineal gland stimulation system of FIG. 1, according to an embodiment of the present disclosure.

FIG. 5 is a flow diagram illustrating a method of use for the pineal gland stimulation system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to dentistry and more particularly to a pineal gland stimulation system as used to improve positive effect of meditation and spirituality through vibration means.

Generally, the ‘Know Thy Self By Vibrations’ is a wireless vibration mechanism resembling a retainer. The device is inserted into the mouth and contours to the roof of the mouth. An internal vibration mechanism can be triggered, allowing the device to vibrate. The vibrations are intended to trigger the pineal gland between the two hemispheres of the brain. A wireless device can be used to control the vibration frequency. Users can choose from various frequencies to produce different vibration effects. The device is intended to create situations that can be used for a variety of positive effects through meditation and spirituality. The apparatus may be constructed using plastic and other suitable materials. The device may be available in various sizes to accommodate all user needs and preferences.

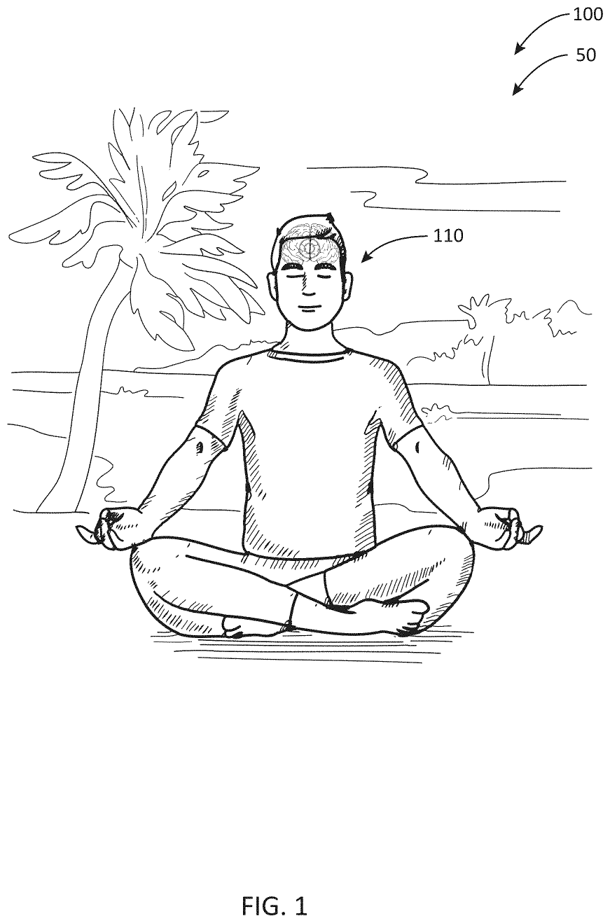

Referring now more specifically to the drawings by numerals of reference, there is shown in FIGS. 1-4, various views of a pineal gland stimulation system 100.

FIG. 1 shows a pineal gland stimulation system 100 during an ‘in-use’ condition 50, according to an embodiment of the present disclosure. Here, the pineal gland stimulation system 100 may be beneficial for use by a user to sooth the user-wearer such that meditation and spiritual awakening durations are maximized. As illustrated, the pineal gland stimulation system 100 may include an oral mouth-piece assembly 110 including a body 112 contoured to fit to the roof of the mouth of the user-wearer having an outer surface 114; and an interior (inner) volume 116; a vibration mechanism 120 in communication with an external electronic device 102 such that signals can be sent between the vibration mechanism 120 and the external electronic device 102 to control vibration frequency of the vibration mechanism 120; and a battery 122 for powering the vibration mechanism 120. As designed, vibrations of the vibration mechanism 120, when activated, trigger the pineal gland of the user-wearer between the two hemispheres of his/her brain. FIG. 1 Shows a user meditating with the present device in his/her mouth. Those with ordinary skill in the art will now appreciate that upon reading this specification and by their understanding the art of communication as described herein, methods of communicating signals between the vibration mechanism 120 and the external electronic device 102 will be understood by those knowledgeable in such art.

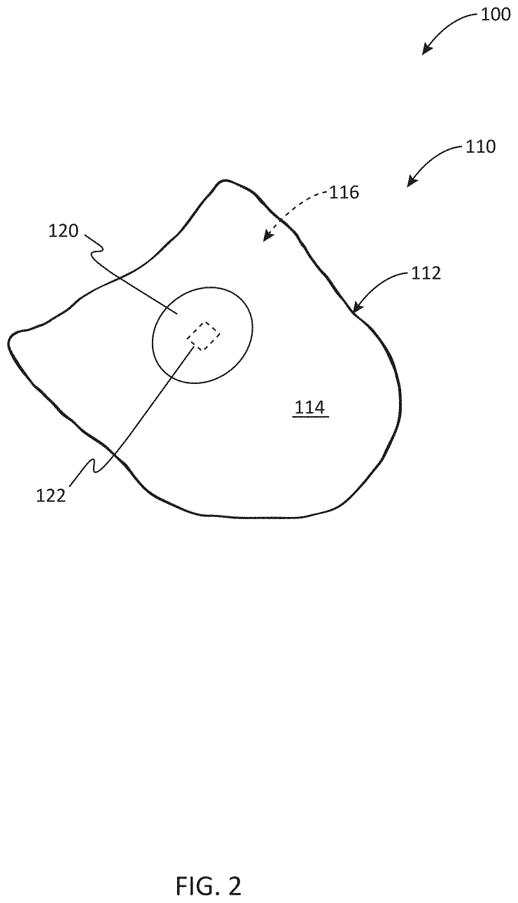

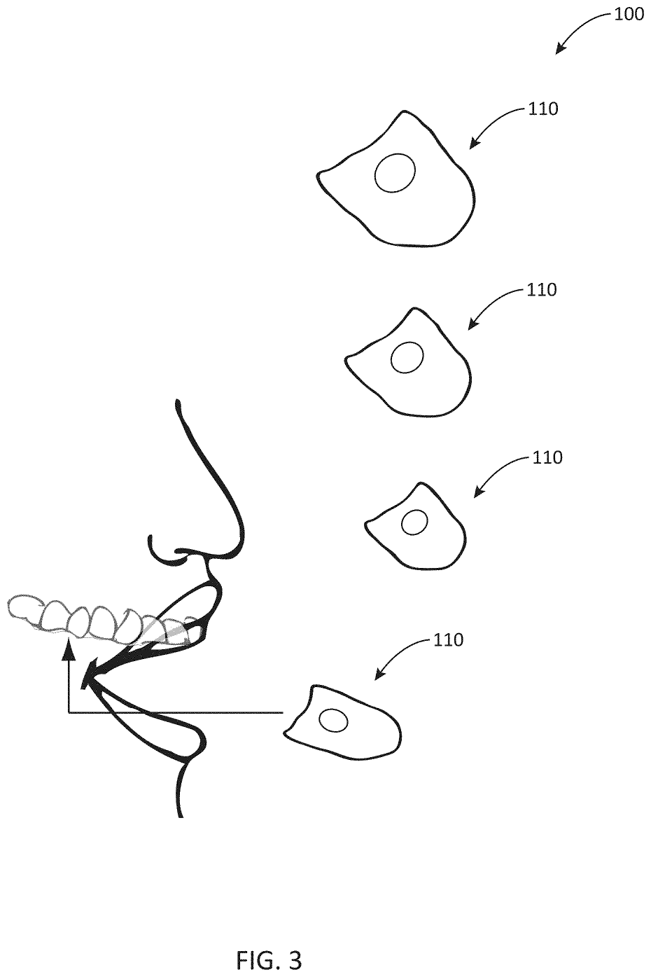

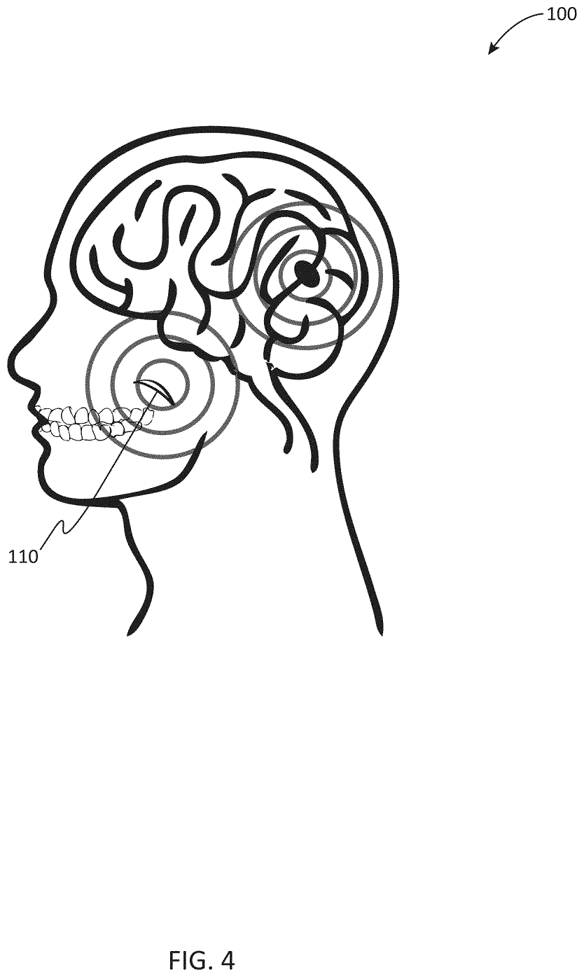

FIG. 2 shows a standalone view of the present invention; FIG. 3 shows how the device contours to the roof of the mouth and is available in various sizes; and FIG. 4 shows how the vibration frequency can target the pineal gland. The vibration frequency is adjustable via the external electronic device 102 and an on/off condition is controlled via the external electronic device 102. Other conditions/optional settings may also be controlled. The battery 122 is located in the interior volume 116 of the body 112 in preferred embodiments; wherein the vibration mechanism 120 and the battery 122 may be integral.

The body 112 is flexible to promote comfort during use; wherein the body 112 is deformable and suctional to the roof of the mouth of the user-wearer. Other mounting or coupling means may be used to hold the oral mouth-piece assembly 110 adjacent the roof of the user's mouth. The oral mouth-piece assembly 110 of the pineal gland stimulation system 100 has a substantially flat profile, as shown. The body 112 may comprise plastic or silicone gel or the like. Those with ordinary skill in the art will now appreciate that upon reading this specification and by their understanding related to the art of materials suitable for human oral use as described herein, methods of making and materials used for the present invention will be understood by those knowledgeable in such art. Other suitably equivalent materials may be used.

As previously mentioned, the outer surface 114 of the body 112 is suction-deformable to couple to the roof of the mouth of the user-wearer; wherein the body 112 has an arcuate-front suitable for placement adjacent (behind) the teeth of the user-wearer. The vibration mechanism 120 and the battery 122 may be non-integral in certain embodiments and battery 122 may be remotely rechargeable. The vibration mechanism 120 is preferably located in the interior volume of the body 112; wherein the vibrations emit from to create and maintain a soothing effect. The oral mouth-piece assembly 110 of the pineal gland stimulation system 100 fits entirely within the mouth of the user-wearer and behind the teeth of the user-wearer such that it is effectively concealed during use.

According to one embodiment, the pineal gland stimulation system 100 may be arranged as a kit. In particular, the pineal gland stimulation system 100 may further include a set of instructions. The instructions may detail functional relationships in relation to the structure of the pineal gland stimulation system 100 such that the pineal gland stimulation system 100 can be used, maintained, or the like, in a preferred manner.

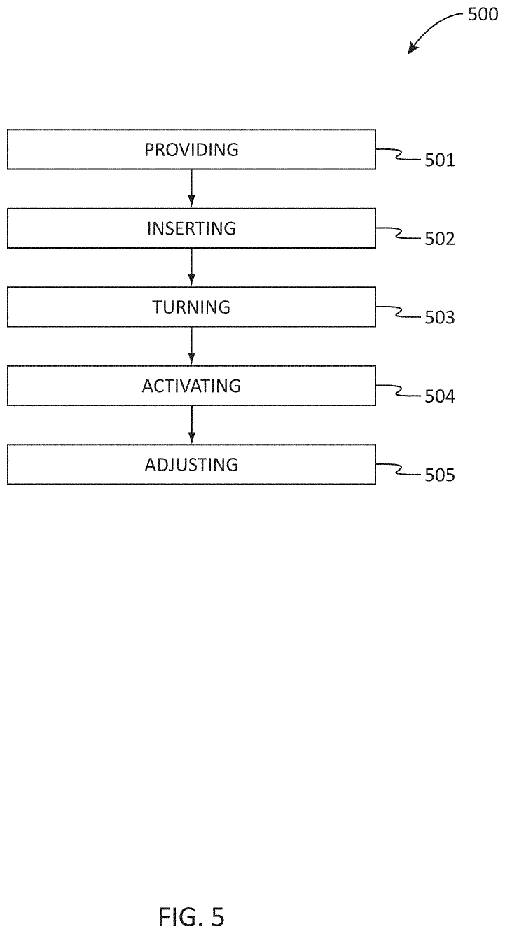

FIG. 5 is a flow diagram illustrating a method of use 500 for the pineal gland stimulation system 100, according to an embodiment of the present disclosure. In particular, the method of use 500 for pineal gland stimulation system 100 may include one or more components or features of the pineal gland stimulation system 100 as described above. As illustrated, the method of use 500 may include the steps of: step one 501, providing an oral mouth-piece assembly as disclosed above; step two 502, inserting the oral mouth-piece assembly into the mouth of the user-wearer behind teeth and on the roof of the mouth; step three 503, turning the vibration mechanism to an on-condition via the external electronic device; then step four 504 activating the vibration mechanism to vibrate, that when activated triggers a pineal gland of the user-wearer between the two hemispheres of the brain of the user-wearer; and next step five 505 adjusting the frequency of vibration via the external electronic device; wherein the oral mouth-piece assembly fits entirely within the mouth of the user-wearer and behind teeth of the user-wearer during use.

The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for using pineal gland stimulation system 100 are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A pineal gland stimulation system, the system comprising:
an oral mouth-piece assembly including
a body contoured to fit to a roof of a mouth of a user-wearer having
an outer surface; and
an interior volume;
a vibration mechanism in communication with an external electronic device such that signals can be sent between said vibration mechanism and said external electronic device to control vibration frequency of said vibration mechanism; and
a battery for powering said vibration mechanism; and
wherein vibrations of said vibration mechanism, when activated, trigger a pineal gland of the user-wearer between two hemispheres of a brain of said user-wearer.

2. The pineal gland stimulation system of claim 1, wherein said vibration frequency is adjustable via said external electronic device.

3. The pineal gland stimulation system of claim 2, wherein said battery is located in said interior volume of said body.

4. The pineal gland stimulation system of claim 2, wherein and on/off condition is controlled via said external electronic device.

5. The pineal gland stimulation system of claim 1, wherein the body is flexible.

6. The pineal gland stimulation system of claim 5, wherein the body is deformable to said roof of said mouth of said user-wearer.

7. The pineal gland stimulation system of claim 3, wherein the vibration mechanism and said battery are integral.

8. The pineal gland stimulation system of claim 1, wherein the oral mouth-piece assembly has a substantially flat profile.

9. The pineal gland stimulation system of claim 1, wherein the body comprises plastic.

10. The pineal gland stimulation system of claim 1, wherein the body comprises silicone gel.

11. The pineal gland stimulation system of claim 6, wherein the outer surface of the body is suction-deformable to couple to said roof of said mouth of said user-wearer.

12. The pineal gland stimulation system of claim 8, wherein the body has an arcuate-front suitable for placement adjacent teeth of said user-wearer.

13. The pineal gland stimulation system of claim 1, wherein the vibration mechanism and said battery are non-integral.

14. The pineal gland stimulation system of claim 2, wherein the vibration mechanism is located in said interior volume of said body.

15. The pineal gland stimulation system of claim 1, wherein the vibrations create a soothing effect.

16. The pineal gland stimulation system of claim 1, wherein the oral mouth-piece assembly fits entirely within the mouth of the user-wearer and behind teeth of said user-wearer.

17. A pineal gland stimulation system, the system comprising:
an oral mouth-piece assembly including
a body contoured to fit to a roof of a mouth of a user-wearer having
an outer surface; and
an interior volume;
a vibration mechanism in communication with an external electronic device such that signals can be sent between said vibration mechanism and said external electronic device to control vibration frequency of said vibration mechanism; and
a battery for powering said vibration mechanism; and
wherein vibrations of said vibration mechanism, when activated, trigger a pineal gland of the user-wearer between two hemispheres of a brain of said user-wearer;
wherein said vibration frequency is adjustable via said external electronic device;
wherein said battery is located in said interior volume of said body;
wherein the vibration mechanism is located in said interior volume of said body;
wherein and on/off condition is controlled via said external electronic device;
wherein the body is flexible;
wherein the body is deformable to said roof of said mouth of said user-wearer;
wherein the body has an arcuate-front suitable for placement adjacent teeth of said user-wearer;
wherein the vibration mechanism and said battery are integral;
wherein the oral mouth-piece assembly has a substantially flat profile; and
wherein the oral mouth-piece assembly fits entirely within the mouth of the user-wearer and behind teeth of said user-wearer.

18. The pineal gland stimulation system of claim 17, further comprising set of instructions; and
wherein the pineal gland stimulation system is arranged as a kit.

19. A method of use for the pineal gland stimulation system, the method comprising the steps of:
providing an oral mouth-piece assembly including
a body contoured to fit to a roof of a mouth of a user-wearer having
an outer surface; and
an interior volume;
a vibration mechanism in communication with an external electronic device such that signals can be sent between said vibration mechanism and said external electronic device to control vibration frequency of said vibration mechanism; and
a battery for powering said vibration mechanism; and
inserting said oral mouth-piece assembly into said mouth of said user-wearer behind teeth and on said roof of said mouth;
turning said vibration mechanism to an on-condition via said external electronic device;
activating said vibration mechanism to vibrate, that when activated triggers a pineal gland of said user-wearer between two hemispheres of a brain of said user-wearer; and
adjusting a frequency of vibration via said external electronic device.

20. The method of claim 19, wherein the oral mouth-piece assembly fits entirely within the mouth of the user-wearer and behind teeth of said user-wearer during use.
